(19) United States
(12) Patent Application Publication
(10) Pub. No.: US 2024/0197832 A1
(43) Pub. Date: Jun. 20, 2024

(54) METHODS AND MATERIALS FOR TREATING A STROKE

(71) Applicants: University of Pittsburgh – Of The Commonwealth System of Higher Education, Pittsburgh, PA (US); THE UNITED STATES GOVERNMENT AS REPRESENTED BY THE DEPARTMENT OF VETERANS AFFAIRS, Washington, DC (US)

(72) Inventors: Xiaoming Hu, Sewickley, PA (US); Jun Chen, Wexford, PA (US); Lawrence R. Wechsler, Pittsburgh, PA (US); Yejie Shi, Pittsburgh, PA (US)

(21) Appl. No.: 18/288,452

(22) PCT Filed: May 24, 2022
(86) PCT No.: PCT/US2022/030657

§ 371 Date: Oct. 26, 2023

Related U.S. Application Data
(60) Provisional application No. 63/192,354, filed on May 24, 2021.

(51) Int. Cl. A61K 38/20; A61K 9/51; A61K 38/17; A61K 38/18; A61K 38/30; A61P 9/10

(52) U.S. Cl. CPC A61K 38/2066; A61K 9/5146; A61K 38/1709; A61K 38/1841; A61K 38/2026; A61K 38/30; A61P 9/10

(57) ABSTRACT

This document relates to methods and materials involved in treating stroke. For example, nanoparticles (e.g., polymer-coated nanoparticles) designed to deliver two or more poly peptides that are normally secreted from mesenchymal stem cells (MSCs; e.g., MSCs of a human less than 33 years of age)) to the brain are provided as well as methods for using such nanoparticles to treat a mammal (e.g., a human) having or having had a stroke.

### CROSS-REFERENCE TO RELATED APPLICATIONS

This application claims the benefit of U.S. Patent Application Ser. No. 63/192,354, filed on May 24, 2021. The disclosure of the prior application is considered part of (and is incorporated by reference in) the disclosure of this application.

### STATEMENT REGARDING FEDERAL FUNDING

This invention was made with government support under I01BX003651-01A2, I01BX003377-01, and 15F-RCS-006 awarded by the U.S. Department of Veterans Affairs. The government has certain rights in the invention.

### TECHNICAL FIELD

This document relates to methods and materials involved in treating a stroke. For example, this document provides nanoparticles (e.g., polymer-coated nanoparticles) designed to deliver two or more polypeptides to the brain to treat a mammal (e.g., a human) having or having had a stroke.

### BACKGROUND INFORMATION

Currently, tissue plasminogen activator (tPA) thrombolysis and mechanical thrombectomy (blood clot retrieval) are the only FDA-approved treatments for ischemic stroke. However, only a small fraction of patients (e.g., <15% of total stroke patients) can benefit from either of these therapies (e.g., Fang et al., J. Hosp. Med., 5:406-409 (2010); Grotta et al., Arch. Neurol., 58:2009 (2001); and Mackenzie et al., World Neurosurg., 138:e839-e846 (2020)). Once brain damage occurs within hours after stroke, no current treatment can stop the process of brain deterioration and the loss of neurological function in stroke patients.

### SUMMARY

This document provides methods and materials involved in treating a stroke. For example, this document provides nanoparticles (e.g., polymer-coated nanoparticles) designed to deliver two or more polypeptides to the brain to treat a mammal (e.g., a human) having or having had a stroke. In some cases, a mammal (e.g., a human) having (e.g., currently experiencing) a stroke can be administered a composition including nanoparticles (e.g., polymer-coated nanoparticles) including two or more polypeptides to treat the mammal. In some cases, a mammal (e.g., a human) having had a stroke can be administered a composition including nanoparticles (e.g., polymer-coated nanoparticles) including two or more polypeptides to treat the mammal.

As demonstrated herein, a panel of polypeptides (e.g., an interleukin (IL)-4 polypeptide, an IL-10 polypeptide, a transforming growth factor beta (TGFß) polypeptide, an insulin-like growth factor (IGF) 1 polypeptide, and a draxin polypeptide) are released at higher levels by therapeutically competent mesenchymal stem cells (MSCs such as human MSCs). Also as described herein, panels of designer secretome (DS) polypeptides (e.g., an IL-4 polypeptide, an IL-10 polypeptide, a TGFß polypeptide, an IGF1 polypeptide, a draxin polypeptide, or combinations thereof) can be used to make polyethylene glycol-coated nanoparticles (PEG-NPs) containing the DS polypeptides (PEG-NP-DS) that can be used to deliver the DS polypeptides to the brain. Administration of PEG-NP-DS including IL-10 polypeptides, TGFß polypeptides, IGF1 polypeptides, and draxin polypeptides can be used to treat stroke. For example, PEG-NP-DS including IL-10 polypeptides, TGFß polypeptides, IGF1 polypeptides, and draxin polypeptides can improve sensorimotor functions, cognitive functions, and emotional functions in a mammal having or having had a stroke.

Having the ability to improve sensorimotor functions, cognitive functions, and emotional functions in a mammal having or having had a stroke as described herein (e.g., by administering nanoparticles such as polymer-coated nanoparticles including two or more polypeptides that are polypeptides normally secreted from therapeutically competent MSCs such as MSCs from humans less than 33 years of age) can provide a non-invasive treatment for stroke that can be used to reduce brain damage and/or to enhance functional recovery in stroke patients.

In general, one aspect of this document features compositions comprising two or more populations of nanoparticles, where each population of nanoparticles independently includes a polypeptide selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide. The nanoparticles of the two or more populations of nanoparticles can be polymer-coated nanoparticles. The polymer-coated nanoparticles can be polyethylene glycol (PEG)-coated nanoparticles. The composition can comprise a first population of nanoparticles including the IL-10 polypeptide, a second population of nanoparticles including the TGFß polypeptide, a third population of nanoparticles including the IGF1 polypeptide, and a fourth population of nanoparticles including the draxin polypeptide. The composition can comprise a first population of nanoparticles including the IL-4 polypeptide, a second population of nanoparticles including the IL-10 polypeptide, a third population of nanoparticles including the TGFß polypeptide, a fourth population of nanoparticles including the IGF1 polypeptide, and a fifth population of nanoparticles including the draxin polypeptide.

In another aspect, this document features compositions comprising a plurality of nanoparticles, where each nanoparticle includes at least two polypeptides selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide. Each nanoparticle can include the IL-10 polypeptide, the TGFß polypeptide, the IGF1 polypeptide, and the draxin polypeptide. Each nanoparticle can include the IL-4 polypeptide, the IL-10 polypeptide, the TGFß polypeptide, the IGF1 polypeptide, and the draxin polypeptide.

In another aspect, this document features methods for treating a mammal having a stroke. The methods can include, or consist essentially of, administering a comprising two or more populations of nanoparticles, where each population of nanoparticles independently includes a polypeptide selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide, and/or administering a composition comprising a plurality of nanoparticles, where each nanoparticle includes at least two polypeptides selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide. The administering can be performed while the mammal is experiencing the stroke. The mammal can be a human. The stroke can be an ischemic stroke, a hemorrhagic stroke, or a ministroke. The method can be effective to reduce or eliminate a symptom of stroke in the mammal. The symptom can be slurring words, confusion, paralysis of the face, arm, or leg, numbness of the face, arm, or leg, weakness of the face, arm, or leg, problems seeing, headache, stumbling, losing balance when walking, dizziness, or loss of coordination. The method can be effective to improve sensorimotor function in the mammal. The method can be effective to improve cognitive function in the mammal. The method can be effective to improve psychiatric function in the mammal.

In another aspect, this document features methods for treating a mammal having had a stroke. The methods can include, or consist essentially of, administering a composition comprising two or more populations of nanoparticles, where each population of nanoparticles independently includes a polypeptide selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide, and/or administering a composition comprising a plurality of nanoparticles, where each nanoparticle includes at least two polypeptides selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide. The administering can be performed within from about 1 minute to about 72 hours of the mammal experiencing the stroke. The mammal can be a human. The stroke can be an ischemic stroke, a hemorrhagic stroke, or a ministroke. The method can be effective to reduce or eliminate a symptom of stroke in the mammal. The symptom can be slurring words, confusion, paralysis of the face, arm, or leg, numbness of the face, arm, or leg, weakness of the face, arm, or leg, problems seeing, headache, stumbling, losing balance when walking, dizziness, or loss of coordination. The method can be effective to improve sensorimotor function in the mammal. The method can be effective to improve cognitive function in the mammal. The method can be effective to improve psychiatric function in the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A and 1B. Transcriptomic analysis of young and aged human bone marrow-derived mesenchymal stem cells. Publicly available RNA sequencing data acquired from the human bone marrow-derived mesenchymal stem cells (MSCs) at passage 1 (young) and 15 (aged) were analyzed (National Center for Biotechnology Information (NCBI) Gene Expression Omnibus (GEO) Accession No. GSE137186). FIG. 1A: A volcano plot visualizing the differentially expressed genes (DEGs) that are significantly upregulated or downregulated in aged MSC. FIG. 1B: A heatmap showing downregulation of trophic factors and immunomodulatory factors in aged MSCs compared to young MSCs. Four factors that were selected for the cocktail treatment complex are highlighted.

FIG. 2. qPCR analysis of expression levels of candidate genes in human mesenchymal stem cells. MSCs from young male donors (YM), young female donors (YF), or aged female donors (AF) were subjected to qPCR for the 12 candidate genes. All data were normalized to the YM group, and expressed as mean±standard deviation (SD), n=4 per group, *p<0.05, **p<0.01, ***p<0.001 between the indicated groups, One-way ANOVA followed by Bonferroni post hoc test. ns=not significant.

FIG. 3. PEG-ASA and FITC-labeled albumin was used as a model to determine the formation of nanoparticles by Size Exclusion Chromatography. Briefly, PD-10 desalting column was filled with Sephadex G-25 medium, which could separate particle size range from 85 to 260 nm. At first, the column was equilibrated with approximately 25 mL PBS buffer. Sample (0.5 mL) was loaded to the column and eluted with 15 mL elution buffer. The flow-through was collected in a 96-well plate at one drop per well. The UV absorption intensity was measured by BIO-RAD™ microplate spectrophotometer.

FIGS. 4A-4C. Characterization of PEG-NPs carrying DS polypeptides. FIG. 4A: The particle size distribution of each PEG-NP-DS was determined by dynamic light scattering. FIG. 4B: Zeta potential was measured to quantify the particle surface charge of each PEG-NP-DA using the Zetassizer 4 (Malvern Instrument, UK). The normal range for PEG-NP is between −20 mV and 20 mV. FIG. 4C: A representative image of transmission electron microscopy on mixed PEG-NP-DS containing each of the 4 DS polypeptides. The image shows homogenous sizes of PEG-NP-DS. Scale bar=50 nm.

FIGS. 5A and 5B. PEG-NP-DS treatment is safe in mice. FIG. 5A: Survival rate up to 35 days after dMCAO. **p<0.01 vs. Sham group. Log-rank test. FIG. 5B: Body weight was measured in all animals up to 35 days after dMCAO. Data are mean±SD. n=14-19 per group, ns=no significant difference. ***p<0.001 vs. sham. Two-way repeated ANOVA followed by Bonferroni post hoc.

FIGS. 6A and 6B. Open field tests in stroke mice and sham nonstroke mice. FIG. 6A: Total distance travelled within 3 minutes of tests. FIG. 6B: Average speed of travels. Data are mean±SD. n=14-19 per group, ns=no significant difference. ***p<0.001 vs. sham group. Two-way repeated ANOVA followed by Bonferroni post hoc tests.

FIGS. 7A-7G. PEG-NP-DS treatment improves sensorimotor functions after stroke in aged mice. FIG. 7A: Illustration of experimental timeline. Animals were randomly assigned to receive intranasal administration of vehicle or PEG-NP-DS (containing IGF-1, TGFβ, IL-10, and draxin), starting at 24 hours and once every two days until 15 days after dMCAO. FIG. 7B: The rotarod test was performed up to 28 days after dMCAO or sham operation. Data are expressed as the latency to fall off the rotating rod. FIG. 7C: The cylinder test was performed up to 28 days after dMCAO or sham operation. Data are expressed as the contralateral forepaw use (%). FIG. 7D-7E: The foot fault tests were performed up to 28 days after dMCAO or sham operation. The data are expressed as the percentages of errors out of total steps recorded. FIG. 7F-7G: The adhesive removal tests were performed up to 28 days after dMCAO or sham operation. Data are expressed as the latency to touch the tape (FIG. 7F) or remove the tape from the impaired right forepaw (FIG. 7G). All data are mean±SD. n=11-16 per group, ns=no significant difference. ***p<0.001, **p<0.05, *p<0.05. Two-way repeated measures ANOVA followed by Bonferroni post hoc tests.

FIGS. 8A-8F. PEG-NP-DS treatment improves cognitive and emotional functions after stroke in aged mice. Experimental timeline was the same as FIG. 7A. Animals were randomly assigned to receive intranasal administration of vehicle or PEG-NP-DS (containing IGF-1,TGFβ, IL-10, and draxin), starting at 24 hours and once every two days until 15 days after dMCAO. FIGS. 8A and 8B: The 8-arm radial maze test was performed before and 35 days after dMCAO or sham operation. Data are expressed as the number of revisiting errors and total distance traveled, respectively. FIGS. 8C and 8D: The Y-maze test was performed before and 35 days after dMCAO or sham operation. Data are expressed as time spent in the new arm and spontaneous alterations (%), respectively. FIG. 8E: The forced swimming test was performed before and 35 days after dMCAO or sham operation. The data are expressed as immobility time (second). FIG. 8F: The tail suspension test was performed before and 35 days after dMCAO or sham operation. The data are expressed as immobility time (second). All data are mean±SD. n=14-19 per group, ns=no significant difference. ***p<0.001, **p<0.05, *p<0.05. ANOVA followed by Bonferroni post hoc tests.

### DETAILED DESCRIPTION

This document provides methods and materials involved in treating a stroke. For example, this document provides nanoparticles (e.g., polymer-coated nanoparticles) designed to deliver two or more polypeptides that are normally secreted from MSCs (e.g., MSCs of a human less than 33 years of age) to the brain to treat a mammal (e.g., a human) having or having had a stroke. For example, this document provides nanoparticles (e.g., polymer-coated nanoparticles) designed to deliver IL-10 polypeptides, TGFβ polypeptides, IGF1 polypeptides, and draxin polypeptides to the brain to treat a mammal (e.g., a human) having or having had a stroke. In some cases, a mammal (e.g., a human) having a stroke can be administered a composition including nanoparticles (e.g., polymer-coated nanoparticles) including two or more polypeptides that are normally secreted from MSCs (e.g., a human MSCs of a human less than 33 years of age) to treat the mammal. For example, a mammal (e.g., a human) having a stroke can be administered a composition including nanoparticles (e.g., polymer-coated nanoparticles) designed to deliver IL-10 polypeptides, TGFβ polypeptides, IGF1 polypeptides, and draxin polypeptides to treat the mammal. In some cases, a mammal (e.g., a human) having had a stroke can be administered a composition including nanoparticles (e.g., polymer-coated nanoparticles) including two or more polypeptides that are normally secreted from MSCs (e.g., MSCs of a human less than 33 years of age) to treat the mammal. For example, a mammal (e.g., a human) having had a stroke can be administered a composition including nanoparticles (e.g., polymer-coated nanoparticles) designed to deliver IL-10 polypeptides, TGFβ polypeptides, IGF1 polypeptides, and draxin polypeptides to treat the mammal.

A nanoparticle described herein (e.g., a polymer-coated nanoparticle) can include one or more (e.g., one, two, three, four, five, or more) polypeptides that are normally secreted from MSCs (e.g., MSCs of a human less than 33 years of age). For example, a nanoparticle described herein (e.g., a polymer-coated nanoparticle) can include one or more (e.g., one, two, three, four, five, or more) polypeptides selected from the group consisting of IL-4 polypeptides, IL-10 polypeptides, TGFβ polypeptides, IGF1 polypeptides, draxin polypeptides, NPTX2 polypeptides, PTN polypeptides, TGFB3 polypeptides, PRXL2A polypeptides, MMP13 polypeptides, MMP9 polypeptides, and ADAM22 polypeptides. In some cases, a nanoparticle described herein can include four polypeptides that are normally secreted from MSCs (e.g., MSCs of a human less than 33 years of age). For example, a nanoparticle described herein can be designed to contain a IL-10 polypeptide, a TGFβ polypeptide, a IGF1 polypeptide, and a draxin polypeptide. In some cases, a nanoparticle described herein can include five polypeptides that are normally secreted from MSCs (e.g., MSCs of a human less than 33 years of age). For example, a nanoparticle described herein can be designed to contain an IL-4 polypeptide, an IL-10 polypeptide, a TGFβ polypeptide, an IGF1 polypeptide, and a draxin polypeptide. Any appropriate IL-10 polypeptide can be used as described herein. For example, a human IL-10 polypeptide can be used as described herein. An example of an IL-10 polypeptide that can be used as described herein includes, without limitation, a polypeptide having the amino acid sequence set forth in National Center for Biotechnology Information (NCBI) Accession No: P22301. Any appropriate TGFβ polypeptide can be used as described herein. For example, a human TGFβ polypeptide can be used as described herein. An example of a TGFβ polypeptide that can be used as described herein includes, without limitation, a polypeptide having the amino acid sequence set forth in NCBI Accession No: P01137. Any appropriate IGF1 polypeptide can be used as described herein. For example, a human IGF1 polypeptide can be used as described herein. An example of an IGF1 polypeptide that can be used as described herein includes, without limitation, a polypeptide having the amino acid sequence set forth in NCBI Accession No: P05019. Any appropriate draxin polypeptide can be used as described herein. For example, a human draxin polypeptide can be used as described herein. An example of a draxin polypeptide that can be used as described herein includes, without limitation, a polypeptide having the amino acid sequence set forth in NCBI Accession No: NP_940947. Any appropriate IL-4 polypeptide can be used as described herein. For example, a human IL-4 polypeptide can be used as described herein. An example of an IL-4 polypeptide that can be used as described herein includes, without limitation, a polypeptide having the amino acid sequence set forth in NCBI Accession No: P05112.

In some cases, two or more polypeptides that are normally secreted from MSCs (e.g., MSCs of a human less than 33 years of age) can be encapsulated (e.g., contained) within a nanoparticle described herein (e.g., a polymer-coated nanoparticle). For example, IL-10 polypeptides, TGFβ polypeptides, IGF1 polypeptides, and draxin polypeptides can be encapsulated (e.g., contained) within a nanoparticle described herein (e.g., a polymer-coated nanoparticle).

In some cases, two or more polypeptides that are normally secreted from MSCs (e.g., MSCs of a human less than 33 years of age) can be conjugated (e.g., covalently or non-covalently conjugated) to a nanoparticle described herein (e.g., a polymer-coated nanoparticle). For example, IL-10 polypeptides, TGFβ polypeptides, IGF1 polypeptides, and draxin polypeptides can be covalently conjugated to a nanoparticle described herein (e.g., a polymer-coated nanoparticle). In some cases, a nanoparticle described herein can have two or more polypeptides that are normally secreted from MSCs (e.g., MSCs of a human less than 33 years of age) covalently conjugated to the nanoparticle. In some cases, a nanoparticle described herein can have two or more polypeptides that are normally secreted from MSCs (e.g., MSCs of a human less than 33 years of age) non-covalently conjugated to the nanoparticle.

Any polypeptide that is normally secreted from MSCs (e.g., MSCs of a human less than 33 years of age) can be included in a nanoparticle described herein (e.g., a polymer-coated nanoparticle). Examples of polypeptides that are normally secreted from MSCs (e.g., MSCs of a human less than 33 years of age) and that can be included in a nanoparticle described herein include, without limitation, IL-4 polypeptides, IL-10 polypeptides, TGFβ polypeptides, IGF1 polypeptides, draxin polypeptides, NPTX2 polypeptides, PTN polypeptides, TGFB3 polypeptides, PRXL2A polypeptides, MMP13 polypeptides, MMP9 polypeptides, and ADAM22 polypeptides. In some cases, a polypeptide that is normally secreted from MSCs (e.g., MSCs of a human less than 33 years of age) and that can be included in a nanoparticle described herein can be a trophic polypeptide. In some cases, a polypeptide that is normally secreted from MSCs (e.g., MSCs of a human less than 33 years of age) and that can be included in a nanoparticle described herein can be an immunomodulatory polypeptide. In some cases, a polypeptide that is normally secreted from MSCs (e.g., MSCs of a human less than 33 years of age) and that can be included in a nanoparticle described herein can be a metalloproteinase. In some cases, a polypeptide that is normally secreted from MSCs (e.g., MSCs of a human less than 33 years of age) and that can be included in a nanoparticle described herein can be as described elsewhere (see, e.g., Wang et al. J Cereb Blood Flow Metab 40(1_suppl): S81-S97 (2020); Jiang et al. J Cereb Blood Flow Metab 40(4): 720-738 (2020); and Shi et al. J Cereb Blood Flow Metab 40(1_suppl):S49-S66 (2020)).

A nanoparticle described herein (e.g., a polymer-coated nanoparticle) can be any appropriate nanoparticle. Examples of nanoparticles that can include two or more polypeptides that are normally secreted from MSCs (e.g., MSCs of a human less than 33 years of age) as described herein include, without limitation, liposomes and exosomes.

A nanoparticle described herein (e.g., a polymer-coated nanoparticle) can be any appropriate size (e.g., can have any appropriate average diameter). For example, a nanoparticle that can include two or more polypeptides that are normally secreted from MSCs (e.g., MSCs of a human less than 33 years of age) as described herein can have a size (e.g., a maximum dimeter) of from about 80 nanometers (nm) to about 300 nm (e.g., from about 80 nm to about 275 nm, from about 80 nm to about 250 nm, from about 80 nm to about 225 nm, from about 80 nm to about 200 nm, from about 80 nm to about 175 nm, from about 80 nm to about 150 nm, from about 80 nm to about 125 nm, from about 80 nm to about 100 nm, from about 100 nm to about 300 nm, from about 125 nm to about 300 nm, from about 150 nm to about 300 nm, from about 175 nm to about 300 nm, from about 200 nm to about 300 nm, from about 225 nm to about 300 nm, from about 250 nm to about 300 nm, from about 100 nm to about 275 nm, from about 125 nm to about 250 nm, from about 150 nm to about 225 nm, from about 175 nm to about 200 nm, from about 100 nm to about 150 nm, from about 150 nm to about 200 nm, or from about 200 nm to about 250 nm).

In some cases, a nanoparticle described herein (e.g., a polymer-coated nanoparticle) can be a polymer-coated nanoparticle. When a nanoparticle described herein is a polymer-coated nanoparticle, the nanoparticle can be coated with any appropriate polymer(s). A “polymer” is a molecule of repeating structural units (e.g., monomers) formed via a chemical reaction, i.e., polymerization. A polymer can be natural polymer or a synthetic polymer. In some cases, a polymer can be a biodegradable polymer. In some cases, a polymer can be a biocompatible polymer. Examples of polymers that can be included in a polymer-coated nanoparticle described herein include, without limitation, PEGs. A polymer can have any appropriate molecular weight (MW; e.g., an average MW). For example, a polymer that can be included in a polymer-coated nanoparticle described herein can have a molecular weight of from about 2000 Daltons (Da) to about 5000 Da. For example, when a polymer is a PEG, the PEG can have a MW (e.g., an average MW) of from about 2000 Da to about 5000 Da (e.g., from about 2000 Da to about 4500 Da, from about 2000 Da to about 4000 Da, from about 2000 Da to about 3500 Da, from about 2000 Da to about 3000 Da, from about 2000 Da to about 2500 Da, from about 2500 Da to about 5000 Da, from about 3000 Da to about 5000 Da, from about 3500 Da to about 5000 Da, from about 4000 Da to about 5000 Da, from about 4500 Da to about 5000 Da, from about 2500 Da to about 4500 Da, for rom about 3000 Da to about 4000 Da). For example, when a polymer is a PEG, the PEG can have a MW of about 3500 Da. Unless otherwise specified, polymer MWs provided herein are weight average MW. In some cases, a polymer can be a copolymer (e.g., can be formed from polymerization of two or more different monomers). When a polymer is a copolymer, the copolymer can be any type of copolymer (e.g., a linear copolymer or a branched copolymer).

In some cases, nanoparticles described herein (e.g., nanoparticles such as polymer-coated nanoparticles including two or more polypeptides that are normally secreted from MSCs) can be formulated into a composition (e.g., a pharmaceutically acceptable compositions). For example, a composition including nanoparticles described herein can include one or more pharmaceutically acceptable carriers (additives), excipients, and/or diluents. Examples of pharmaceutically acceptable carriers, excipients, and diluents that can be used in a composition described herein include, without limitation, phosphate-buffered saline, sucrose, lactose, starch (e.g., starch glycolate), cellulose, cellulose derivatives (e.g., modified celluloses such as microcrystalline cellulose and cellulose ethers like hydroxypropyl cellulose (HPC) and cellulose ether hydroxypropyl methylcellulose (HPMC)), xylitol, sorbitol, mannitol, gelatin, polymers (e.g., polyvinylpyrrolidone (PVP), crosslinked polyvinylpyrrolidone (crospovidone), carboxymethyl cellulose, polyethylene-polyoxypropylene-block polymers, and crosslinked sodium carboxymethyl cellulose (croscarmellose sodium)), titanium oxide, azo dyes, silica gel, fumed silica, talc, magnesium carbonate, vegetable stearin, magnesium stearate, aluminum stearate, stearic acid, antioxidants (e.g., vitamin A, vitamin E, vitamin C, retinyl palmitate, and selenium), citric acid, sodium citrate, parabens (e.g., methyl paraben and propyl paraben), petrolatum, dimethyl sulfoxide, mineral oil, serum polypeptides (e.g., human serum albumin), glycine, sorbic acid, potassium sorbate, water, salts or electrolytes (e.g., saline, protamine sulfate, disodium hydrogen phosphate, potassium hydrogen phosphate, sodium chloride, and zinc salts), colloidal silica, magnesium trisilicate, polyacrylates, waxes, wool fat, and lecithin. A pharmaceutical composition can be formulated for administration in solid or liquid form including, without limitation, sterile solutions, suspensions, sustained-release formulations, tablets, capsules, pills, powders, and granules.

In some cases, a composition including nanoparticles described herein (e.g., nanoparticles such as polymer-coated nanoparticles including two or more polypeptides that are normally secreted from MSCs) can include a plurality of identical nanoparticles that are designed to include two or more particular polypeptides described herein.

In some cases, a composition including nanoparticles described herein (e.g., nanoparticles such as polymer-coated nanoparticles including two or more polypeptides that are normally secreted from MSCs) can include a population of two or more (e.g., two, three, four, five, or more) different nanoparticles. For example, a composition can be designed to include two populations of nanoparticles, with the first population containing TGFβ and IL-polypeptides and the second population containing draxin and IGF-1 polypeptides. For example, a composition can be designed to include two populations of nanoparticles, with the first population containing TGFβ and draxin polypeptides and the second population containing IL-10 and IGF-1 polypeptides. For example, a composition can be designed to include two populations of nanoparticles, with the first population containing TGFβ and IGF-1 polypeptides and the second population containing IL-10 and draxin polypeptides. For example, a composition can be designed to include two populations of nanoparticles, with the first population containing TGFβ, IL-10, and draxin polypeptides and the second population containing IGF-1 and IL-4 polypeptides. For example, a composition can be designed to include two populations of nanoparticles, with the first population containing TGFβ and IL-10 polypeptides and the second population containing draxin, IGF-1, and IL-4 polypeptides.

In some cases, a composition including nanoparticles described herein (e.g., nanoparticles such as polymer-coated nanoparticles including two or more polypeptides that are normally secreted from MSCs) can be designed to include populations of different nanoparticles where each population of nanoparticles of the composition contains a single polypeptide. For example, a composition of nanoparticles can be designed to include a first population of nanoparticles having TGFβ polypeptides, a second population of nanoparticles having IL-10 polypeptides, a third population of nanoparticles having draxin polypeptides, and a fourth population of nanoparticles having IGF-1 polypeptides. In another example, a composition of nanoparticles can be designed to include a first population of nanoparticles having TGFβ polypeptides, a second population of nanoparticles having IL-10 polypeptides, a third population of nanoparticles having draxin polypeptides, a fourth population of nanoparticles having IGF-1 polypeptides, and a fifth population of nanoparticles having IL-4 polypeptides.

In some cases, a composition including nanoparticles described herein (e.g., nanoparticles such as polymer-coated nanoparticles including two or more polypeptides that are normally secreted from MSCs) can be formulated as a delivery system. For example, a composition including nanoparticles described herein can be formulated as a controlled-release delivery system for the two or more polypeptides. Examples of types of controlled-release delivery that a composition including nanoparticles described herein can be formulated for include, without limitation, burst release, slow release, delayed release, and sustained release.

This document also provides methods and materials for using nanoparticles described herein (e.g., nanoparticles such as polymer-coated nanoparticles including two or more polypeptides that are normally secreted from MSCs). In some cases, a mammal (e.g., a human) having a stroke can be treated by administering nanoparticles described herein to the mammal. For example, a mammal currently experiencing a stroke can be administered a composition including nanoparticles described herein to treat the mammal. In some cases, a mammal (e.g., a human) having had a stroke can be treated by administering nanoparticles described herein to the mammal. For example, a mammal having already experienced a stroke can be administered a composition including nanoparticles described herein to treat the mammal.

Any type of mammal having or having had a stroke can be treated using the methods and materials described herein (e.g., by administering nanoparticles such as polymer-coated nanoparticles including two or more polypeptides that are normally secreted from MSCs). Examples of mammals that can be treated as described herein include, without limitation, humans, non-human primates such as monkeys, dogs, cats, horses, cows, pigs, sheep, rabbits, mice, and rats. In some cases, a human having or having had a stroke can be treated by administering nanoparticles (e.g., polymer-coated nanoparticles) including two or more polypeptides that are normally secreted from MSCs (e.g., MSCs of a human less than 33 years of age).

In some cases, methods for treating a mammal (e.g., a human) having or having had a stroke can include identifying that mammal as having or as having had a stroke. For example, clinical history, physical examinations (e.g., to check blood pressure), neurological examinations, blood tests (e.g., to check blood clotting time, to check blood sugar, and to check for infection), and/or imaging (e.g., to look for bleeding in the brain such as computerized tomography (CT) scanning, computerized tomography angiography, magnetic resonance imaging (MRI), carotid ultrasound, and cerebral angiogram) can be used to identify a mammal as having or as having had a stroke. Once identified as having or as having had a stroke, the mammal can be administered or instructed to self-administer nanoparticles described herein (e.g., nanoparticles such as polymer-coated nanoparticles including two or more polypeptides that are normally secreted from MSCs).

A mammal (e.g., a human) having or having had any type of stroke can be treated as described herein (e.g., by administering nanoparticles such as polymer-coated nanoparticles including two or more polypeptides that are normally secreted from MSCs). Examples of types of strokes that can be treated using the methods and materials described herein include, without limitation, ischemic stroke (e.g., large vessel, small vessel, embolic, and crytopgenic ischemic stroke), hemorrhagic stroke (e.g., intraparenchymal, subdural, epidual, and subarachnoid hemorrhagic stroke), and ministroke (transient ischemic attack (TIA)).

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) to reduce or eliminate one or more symptoms of stroke in that mammal. Examples of symptoms of stroke that can be reduced or eliminated using the methods and materials described herein include, without limitation, trouble speaking (e.g., slurring words), trouble understanding what others are saying (e.g., confusion), paralysis (e.g., paralysis of the face, arm, and/or leg on one side), numbness (e.g., numbness of the face, arm, and/or leg on one side), weakness (e.g., weakness of the face, arm, and/or leg on one side such as drooping of one side of mouth when smiling), problems seeing (e.g., in one or both eyes), headache, trouble walking (e.g., stumbling or losing balance when walking), dizziness, and loss of coordination. For example, a composition including nanoparticles described herein can be administered to a mammal (e.g., a human) having or having had a stroke as described herein to reduce one or more symptoms of stroke in the mammal by, for example, 10, 20, 30, 40, 50, 60, 70, 80, 90, 95, or more percent.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) to improve sensorimotor function in that mammal. For example, a composition including nanoparticles described herein can be administered to a mammal (e.g., a human) having or having had a stroke as described herein to improve sensorimotor of stroke in the mammal by, for example, 10, 20, 30, 40, 50, 60, 70, 80, 90, 95, or more percent.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) to improve cognitive function in that mammal. For example, a composition including nanoparticles described herein can be administered to a mammal (e.g., a human) having or having had a stroke as described herein to improve cognitive of stroke in the mammal by, for example, 10, 20, 30, 40, 50, 60, 70, 80, 90, 95, or more percent.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) to improve psychiatric function in that mammal. For example, a composition including nanoparticles described herein can be administered to a mammal (e.g., a human) having or having had a stroke as described herein to improve psychiatric of stroke in the mammal by, for example, 10, 20, 30, 40, 50, 60, 70, 80, 90, 95, or more percent.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) to reduce or eliminate emotional deficits in that mammal. For example, a composition including nanoparticles described herein can be administered to a mammal (e.g., a human) having or having had a stroke as described herein to reduce emotional deficits of stroke in the mammal by, for example, 10, 20, 30, 40, 50, 60, 70, 80, 90, 95, or more percent.

Any appropriate method can be used to administer nanoparticles described herein (e.g., nanoparticles such as polymer-coated nanoparticles including two or more polypeptides that are normally secreted from MSCs) to a mammal (e.g., a human) having or having had a stroke. For example, a composition including nanoparticles described herein can be designed for oral or parenteral (including, without limitation, a subcutaneous, intramuscular, intravenous, intradermal, intra-cerebral, intrathecal, or intraperitoneal (i.p.) injection) administration to a mammal having or having had a stroke. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

In some cases, nanoparticles described herein (e.g., nanoparticles such as polymer-coated nanoparticles including two or more polypeptides that are normally secreted from MSCs) can cross the blood brain barrier. For example, a composition including nanoparticles described herein, when administered to a mammal (e.g., a human), can cross the blood brain barrier and enter the brain of that mammal thereby delivering two or more polypeptides that are normally secreted from MSCs (e.g., MSCs of a human less than 33 years of age) to the brain of that mammal. In some cases, a composition including nanoparticles described herein, when administered to a mammal (e.g., a human), can cross the blood brain barrier and enter the brain of that mammal thereby delivering two or more polypeptides that are normally secreted from MSCs to microglia, macrophages, dendritic cells, oligodendrocytes, cerebrovascular endothelial cells, pericytes, astrocytes, and/or neural stem cells within the brain of that mammal.

Nanoparticles described herein (e.g., nanoparticles such as polymer-coated nanoparticles including two or more polypeptides that are normally secreted from MSCs) can be administered to a mammal (e.g., a human) having or having had a stroke at any appropriate time. For example, a composition containing nanoparticles described herein can be administered to a mammal having a stroke while the mammal is still experiencing the stroke. In some cases, a composition containing nanoparticles described herein can be administered to a mammal having had a stroke within from about 1 minute to about 72 hours (e.g., within from about 1 minute to about 48 hours, within from about 1 minute to about 36 hours, within from about 1 minute to about 24 hours, within from about 1 minute to about 18 hours, within from about 1 minute to about 12 hours, within from about 1 minute to about 6 hours, within from about 1 minute to about 1 hour, within from about 1 hour to about 72 hours, within from about 6 hours to about 72 hours, within from about 12 hours to about 72 hours, within from about 18 hours to about 72 hours, within from about 24 hours to about 72 hours, within from about 36 hours to about 72 hours, within from about 48 hours to about 72 hours, within from about 1 hour to about 48 hours, within from about 6 hours to about 36 hours, within from about 12 hours to about 24 hours, within from about 6 hours to about 12 hours, within from about 12 hours to about 18 hours, within from about 18 hours to about 24 hours, within from about 24 hours to about 36 hours, or within from about 36 hours to about 48 hours) of the mammal experiencing the stroke.

Nanoparticles described herein (e.g., nanoparticles such as polymer-coated nanoparticles including two or more polypeptides that are normally secreted from MSCs) can be administered to a mammal (e.g., a human) having or having had a stroke in any appropriate amount (e.g., any appropriate dose). For example, an effective amount of a composition containing nanoparticles described herein can be any amount that can treat a mammal having or having had a stroke as described herein without producing significant toxicity to the mammal. For example, an effective amount of nanoparticles described herein can include from about 0.45 milligrams per kilogram body weight of the mammal (mg/kg) to about 0.55 mg/kg of TGFβ polypeptides. For example, an effective amount of nanoparticles described herein can include from about 0.45 mg/kg to about 0.55 mg/kg of IL-4 polypeptides. For example, an effective amount of nanoparticles described herein can include from about 0.45 mg/kg to about 0.55 mg/kg of IL-10 polypeptides. For example, an effective amount of nanoparticles described herein can include from about 0.11 mg/kg to about 0.13 mg/kg of IGF1 polypeptides. For example, an effective amount of nanoparticles described herein can include from about 0.08 mg/kg to about 0.12 mg/kg of draxin polypeptides. In some cases, an effective amount of nanoparticles described herein can include from about 0.45 mg/kg to about 0.55 mg/kg of TGFβ polypeptides, from about 0.45 mg/kg to about 0.55 mg/kg of IL-10 polypeptides, from about 0.11 mg/kg to about 0.13 mg/kg of IGF1 polypeptides, and from about 0.08 mg/kg to about 0.12 mg/kg of draxin polypeptides. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the stroke may require an increase or decrease in the actual effective amount administered.

Nanoparticles described herein (e.g., nanoparticles such as polymer-coated nanoparticles including two or more polypeptides that are normally secreted from MSCs) can be administered to a mammal (e.g., a human) having or having had a stroke in any appropriate frequency. The frequency of administration can be any frequency that can treat a mammal having or having had a stroke without producing significant toxicity to the mammal. For example, the frequency of administration can be from about twice a day to about once a day, from about once a day to once every two days, or from about once every 12 hours to about once every 24 hours. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

Nanoparticles described herein (e.g., nanoparticles such as polymer-coated nanoparticles including two or more polypeptides that are normally secreted from MSCs) can be administered to a mammal (e.g., a human) having or having had a stroke for any appropriate duration. An effective duration for administering or using a composition containing nanoparticles described herein can be any duration that can treat a mammal having or having had a stroke without producing significant toxicity to the mammal. For example, the effective duration can vary from several minutes to several hours, from several hours to several days, from several days to several weeks. Multiple factors can influence the actual effective duration used for a particular treatment. For example, an effective duration can vary with the frequency of administration, effective amount, use of multiple treatment agents, and/or route of administration.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) as the sole active ingredient used to the treat the mammal.

In some cases, nanoparticles described herein (e.g., nanoparticles such as polymer-coated nanoparticles including two or more polypeptides that are normally secreted from MSCs) can be administered to a mammal in need thereof (e.g., a mammal such as a human that is having or has had a stroke) together with one or more additional agents and/or therapies used to treat stroke. Examples of additional agents that can be used to treat stroke in combination with the nanoparticles described herein include, without limitation, tPA, blood-thinning medications (e.g., aspirin, clopidogrel, heparin, and warfarin), blood pressure controlling medications, blood sugar controlling medications, and cholesterol lowing medications (e.g. statin). In cases where nanoparticles described herein are used in combination with additional agents used to treat stroke, the one or more additional agents can be administered at the same time (e.g., in a single composition containing both nanoparticles described herein and the one or more additional agents) or independently. For example, nanoparticles described herein can be administered first, and the one or more additional agents administered second, or vice versa. Examples of therapies that can be used to treat stroke in combination with the nanoparticles described herein include, without limitation, removal of a clot with a stent retriever, carotid endarterectomy, angioplasty (e.g., angioplasty without stent insertion and angioplasty with stent insertion), physical therapy, occupational therapy, recreational therapy, and speech therapy. In cases where nanoparticles described herein are used in combination with one or more additional therapies used to treat stroke, the one or more additional therapies can be performed at the same time or independently of the administration of nanoparticles described herein. For example, the nanoparticles described herein can be administered before, during, or after the one or more additional therapies are performed.

The invention will be further described in the following examples, which do not limit the scope of the invention described in the claims.

### EXAMPLES

### Example 1: Designer Secretomes to Protect and Repair the Brain During and after Stroke

### Protective Elements in MSCs

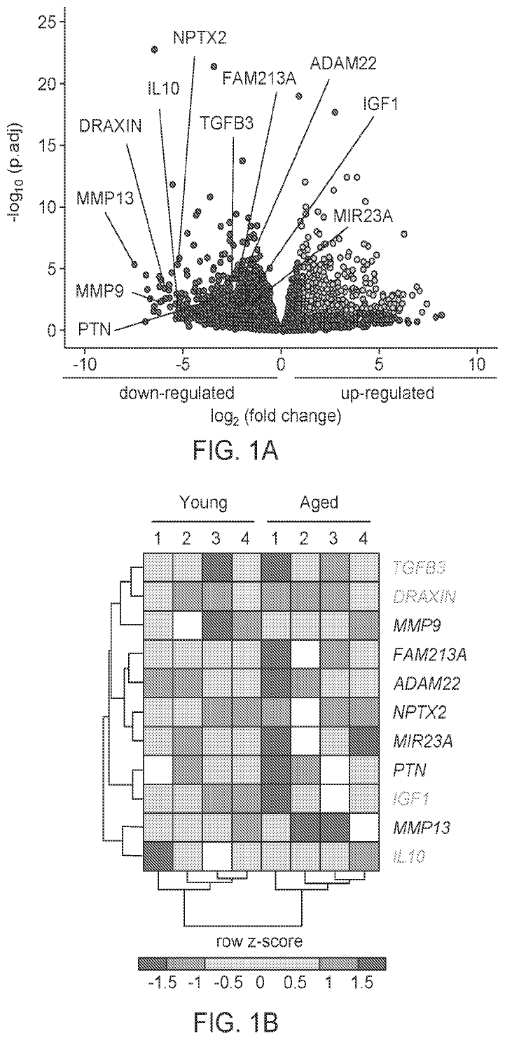

Bibliometric analyses of preclinical research that investigated MSC-related therapies were conducted. First, bioinformatic analysis was performed on a publicly available database (NCBI GEO Accession No. GSE137186) to identify protective elements that were enriched in young MSCs. The data in Year 1 have shown that treatment with MSCs from young human donors is robustly more effective in improving long-term stroke outcomes than MSCs from aged donors. Comparison between young and aged MSCs revealed that a number of gene products are significantly downregulated in aged MSCs compared to young MSCs (FIG. 1), including trophic factors (e.g., DRAXIN, NPTX2, PTN, and IGF1), immunomodulatory factors (e.g., IL10, TGFB3, and PRXL2A), and metalloproteinases (e.g., MMP13, MMP9, and ADAM22).

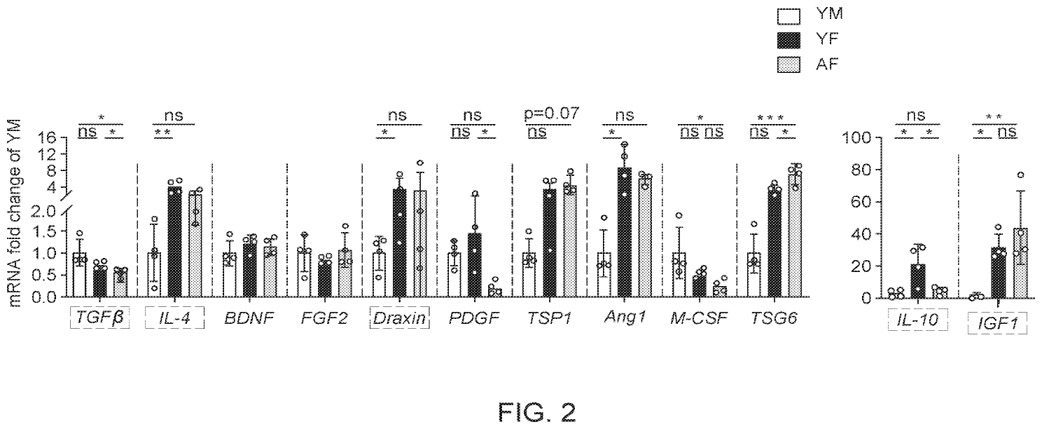

Based on the bioinformatics data, twelve genes were selected for further validation for mRNA expression in MSCs that were derived from either young donors or aged donors. Secretory protective factors that are known to be released from human MSCs, including IL10, TGFB, IGF1, IL-4, BDNF, FGF2, PDGF, TSP1, ANG1, M-CSF, and TSG6 were initially selected. Quantitative RT-PCR (qPCR) was performed to compare the expression levels of candidate protective factors between young and aged MSCs (FIG. 2). These assays identified five factors that are expressed at significantly higher levels in young female MSCs compared to either young male MSCs (IL-4, IL-10, IGF1 and draxin) or aged female MSCs (TGFB and IL-10). These factors were selected as the “designer secretomes” (DS) to make a cocktail treatment complex.

Among the five selected factors for DS (e.g., TGFB, draxin, IL-10, IL-4, and IGF1), IL-10 is a secretory anti-inflammatory cytokine capable of regulating immune responses towards tissue protection and repair. Treatment with IL-10 overexpressed MSCs improves functional recovery after experimental traumatic brain injury by suppressing the production of proinflammatory cytokines from activated microglia/macrophages. TGFβ released from MSCs inhibits the production of pro-inflammatory cytokines in activated microglia via blocking the NF-κB signaling pathway. Draxin is a secretory protein that regulates corpus callosum development via stimulating the production and release of axonal guidance molecules and repelling neurite outgrowth from cortical explants. Draxin also regulates hippocampal neurogenesis in the postnatal dentate gyrus. IL-4 is the best known secretory anti-inflammatory cytokine that transforms microglial and macrophage responses towards an anti-inflammatory and pro-repair phenotype.

### Designer Secretomes for Brain Delivery

Polyethylene glycol-coated nanoparticles (PEG-NPs) were evaluated as a carrier to deliver DS polypeptides across the blood-brain barrier and into stroke brain.

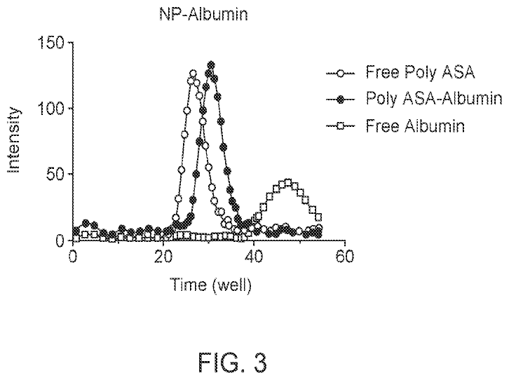

PEG-NPs containing FITC-labeled albumin (FA) were made. The PEG3.5K-FA was synthesized as described elsewhere (see, e.g., Zhang et al., Biomaterials, 31:6075-6086 (2010)). PEG3.5K-FA (78 mg, 0.02 mmol), PEG-b4 PNHS (303 mg, 1 mmol NHS), and TEA (27.5 μL, 0.1 mmol) were then dissolved in DMSO (10 mL) and stirred at 37° C. for 48 hours. Then, 5-ASA (459 mg, 3 mmol) and TEA (416.2 μL, 3 mmol) were added, and the reaction mixture was stirred for another 48 hours. The mixture was dialyzed against DMSO for two days, followed by dialysis against water for three days. The PEG-FASA polymer was obtained after lyophilization. Results of Size Exclusion Chromatography suggested that the PEG-NPs were in the expected size range (FIG. 3).

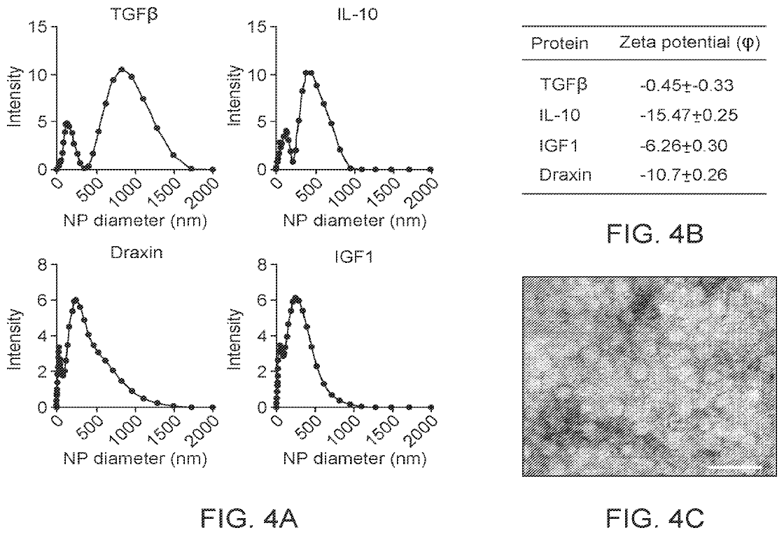

Using the methodology described above, PEG-NPs carrying each of the 4 DS polypeptides (PEG-NP carrying TGFβ polypeptides, PEG-NP carrying IL-10 polypeptides, PEG-NP carrying draxin polypeptides, PEG-NP carrying IGF-1 polypeptides, and PEG-NP carrying IL-4 polypeptides) were produced. The basic physical properties of PEG-NP-TGFβ, PEG-NP-IL-10, and PEG-NP-draxin were characterized (FIG. 4). The results suggested that all parameters appear to be in normal ranges. The concentration of each PEG-NP-DS is: PEG-NP-TGFB (0.8 μg/μL), PEG-NP-IL-10 (0.8 μg/μL), PEG-NP-IGF1 (2 μg/μL), and PEG-NP-draxin (0.16 μg/μL).

### PEG-NP-DS in Stroke Mice

The safety and efficacy of PEG-NP-DS in aged (18 months old) C57/BL6 mice that were subjected to dMCAO-induced stroke was determined. Vehicle or PEG-NP-DS were administrated through the intranasal route in randomly assigned mice starting at 24 hours after stroke. The PEG-NP-DS mixture contained the following components: TGFβ (50 μg/kg), IL-10 (50 μg/kg), IGF1 (120 μg/kg), and draxin (10 μg/kg). The doses for TGFβ, IL-10 and IGF1 were calculated based on doses used in human per m² body surface area (the surface area of a 70 kg human is about 2 m²), and then determined the equivalent doses for mice (90 cm²/mouse).

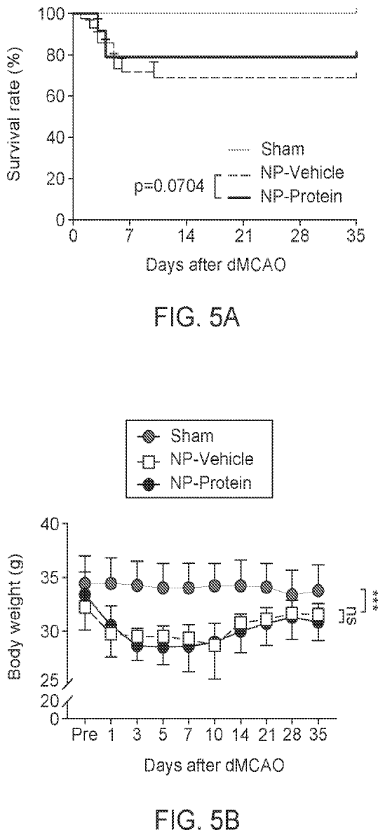

The first set of data obtained included the animal survival curve and body weight curve over 28 days after stroke (FIG. 5). A total of 80 mice entered the study, in which 14 of 14 mice in the sham group, 29 of 42 mice in the NP-Vehicle group, and 19 of 24 mice in the NP-Protein group survived the 35-day testing period. As compared to sham control mice, approximately 30% mice treated with vehicle and about 20% mice treated with PEG-NP-DS mixture were lost (FIG. 5A); there was a trend that PEG-NP-DS-treated mice have a better survival rate after stroke (p=0.074). Both stroke groups showed decreased body weight (FIG. 5B), and no differences were found between vehicle treatment and PEG-NP-DS treatment. These results suggest that treatment with PEG-NP-DS is safe in mice.

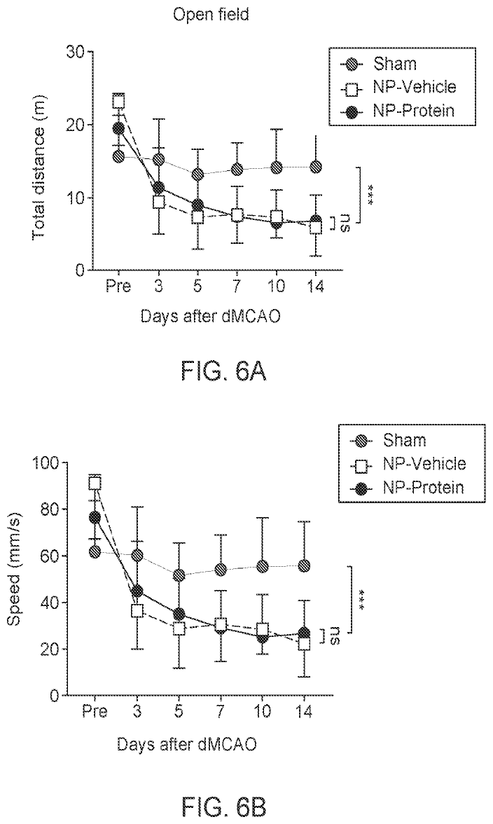

The locomotor functions were measured using the open field test prior to and up to 14 days after stroke or sham operation. Total travel distances and average speed of travels were significantly decreased in both stroke groups compared to the sham group (FIG. 6). No differences were seen between the vehicle-treated and PEG-NP-DS-treated stroke groups. These results demonstrate that PEG-NP-DS treatment is safe in stroke mice.

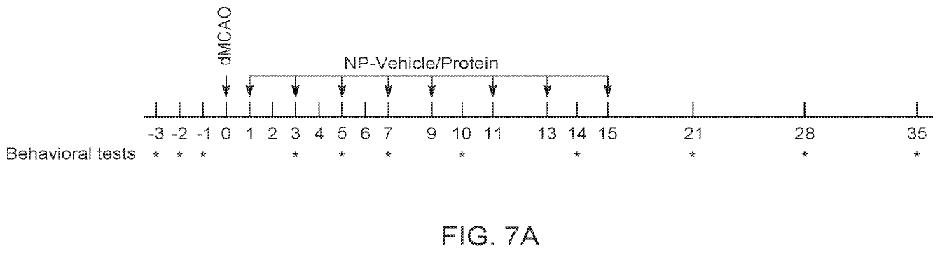

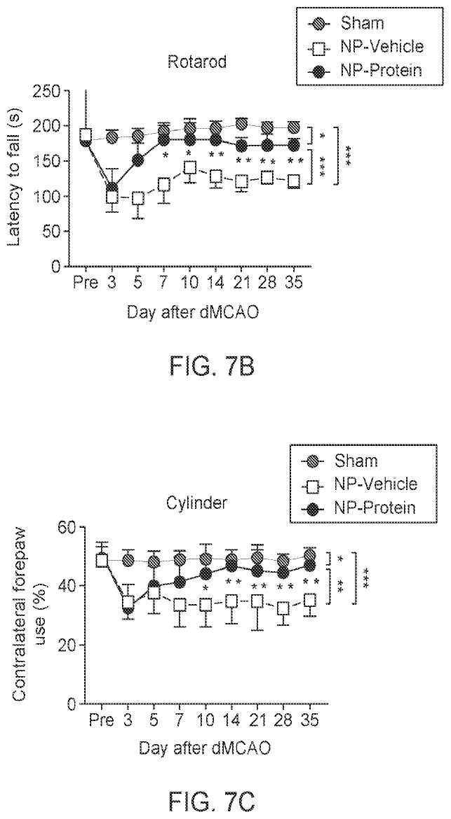

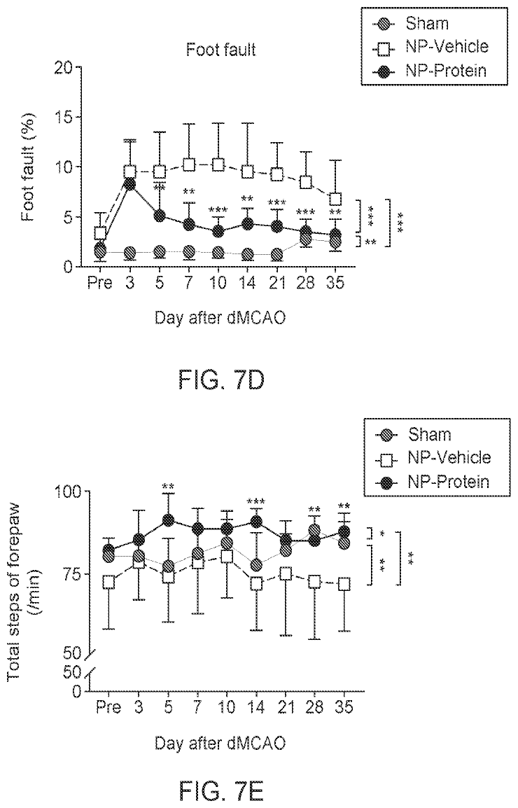

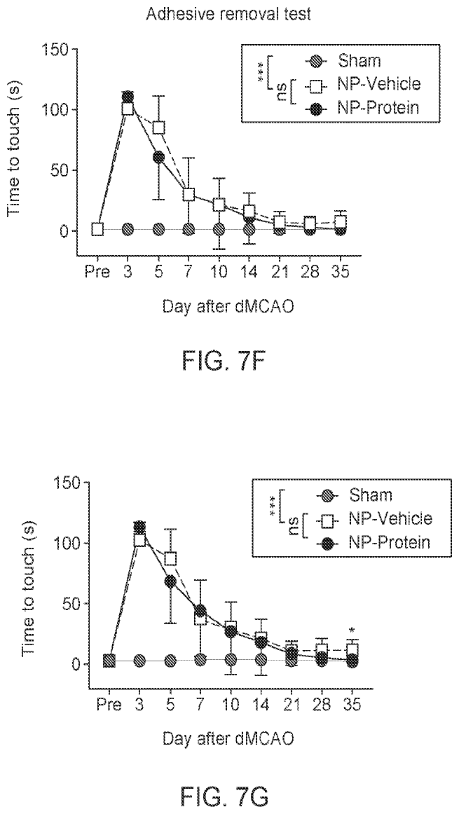

To test the efficacy of PEG-NP-DS in the dMCAO stroke model, mice were randomly assigned to stroke and sham control groups. Equal volumes of PEG-NP-DS or vehicle were administrated through the intranasal route to mice starting at 24 hours after stroke or sham operation and then once every 48 hours until 15 days after surgery (FIG. 7A). A battery of sensorimotor behavioral tests was performed in all mice up to 35 days after stroke or sham operation, including the rotarod test, cylinder test, foot fault test, and adhesive removal test (FIG. 7B-G). All outcome assessments were conducted in a blinded manner. These results showed that post-stroke treatment with PEG-NP-DS significantly improved long term sensorimotor functions of stroke mice in the rotarod test, the cylinder test, and the foot fault test.

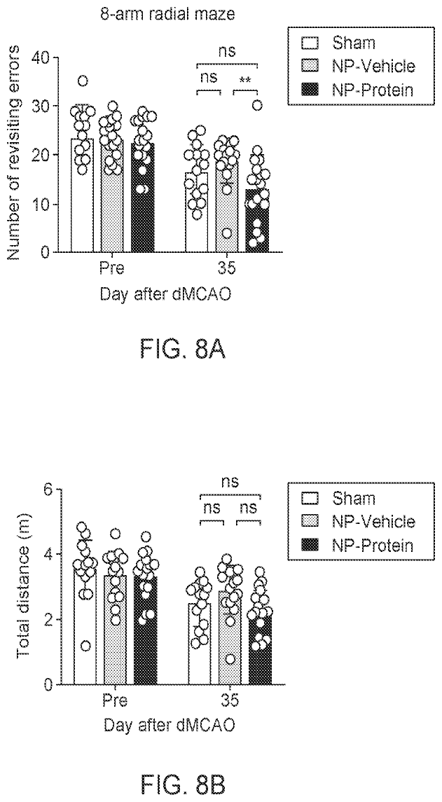

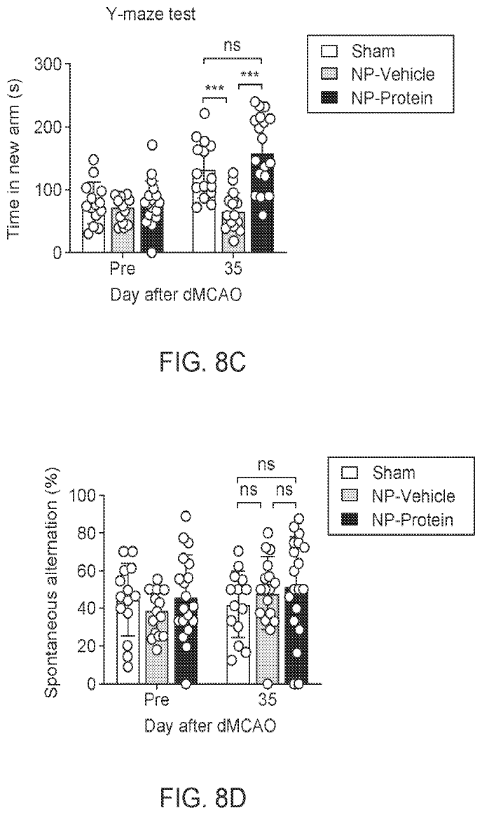

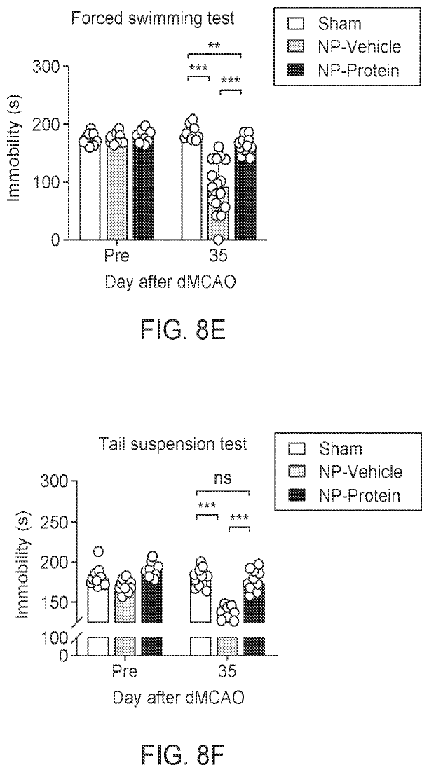

To test the effects of PEG-NP-DS on cognitive and emotional functions after stroke, mice were randomly assigned to stroke and sham control groups. Equal volumes of PEG-NP-DS or vehicle were administrated through the intranasal route to mice starting at 24 hours after stroke or sham operation and then once every 48 hours until 15 days after surgery (FIG. 7A). A battery of behavioral tests was performed in all mice before stroke or at 35 days after stroke or sham operation, including the 8-arm radial maze test, the Y-maze test, the forced swimming test, and the tail suspension test (FIG. 8A-F). All outcome assessments were conducted in a blinded manner. The results showed that post-stroke treatment with PEG-NP-DS significantly improved the long term cognitive performance of stroke mice in the 8-arm radial maze test (FIG. 8A) and the Y-maze test (FIG. 8C) and significantly reduced the emotional deficits of stroke mice in the forced swimming test (FIG. 8E) and the tail suspension test (FIG. 8F).

Cognitive and emotional functions were also assessed using the Morris water maze test (before and 22-27 days after dMCAO or sham operation), the passive avoidance test (before and 35 days after dMCAO or sham operation), the novel object recognition test (before and 14 and 28 days after dMCAO or sham operation), and the elevated plus maze test (before and 35 days after dMCAO or sham operation). However, none of the above tests showed significant differences between vehicle-treated and PEG-NP-DS-treated stroke mice.

### Example 2: Treating a Human Having a Stroke

A human identified as experiencing a stroke is administered a composition containing polymer-coated nanoparticles including from about 0.45 mg/kg to about 0.55 mg/kg of TGFβ polypeptides, from about 0.45 mg/kg to about 0.55 mg/kg of IL-10 polypeptides, from about 0.11 mg/kg to about 0.13 mg/kg of IGF1 polypeptides, and from about 0.08 mg/kg to about 0.12 mg/kg of draxin polypeptides during the stroke. The administered composition can reduce the severity of one or more symptoms of stroke in the mammal.

### Example 3: Treating a Human Having had a Stroke

A human identified as having experienced a stroke is administered a composition containing polymer-coated nanoparticles including from about 0.45 mg/kg to about 0.55 mg/kg of TGFβ polypeptides, from about 0.45 mg/kg to about 0.55 mg/kg of IL-10 polypeptides, from about 0.11 mg/kg to about 0.13 mg/kg of IGF1 polypeptides, and from about 0.08 mg/kg to about 0.12 mg/kg of draxin polypeptides within about 72 hours of the stroke. The administered composition can reduce the severity of one or more symptoms of stroke in the mammal.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising two or more populations of nanoparticles, wherein each population of nanoparticles independently comprises a polypeptide selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide.

2. The composition of claim 1, wherein the nanoparticles of said two or more populations of nanoparticles are polymer-coated nanoparticles.

3. The composition of claim 2, wherein said polymer-coated nanoparticles are polyethylene glycol (PEG)-coated nanoparticles.

4. The composition of claim 1, wherein said composition comprises a first population of nanoparticles comprising said IL-10 polypeptide, a second population of nanoparticles comprising said TGFß polypeptide, a third population of nanoparticles comprising said IGF1 polypeptide, and a fourth population of nanoparticles comprising said draxin polypeptide.

5. The composition of claim 1, wherein said composition comprises a first population of nanoparticles comprising said IL-4 polypeptide, a second population of nanoparticles comprising said IL-10 polypeptide, a third population of nanoparticles comprising said TGFß polypeptide, a fourth population of nanoparticles comprising said IGF1 polypeptide, and a fifth population of nanoparticles comprising said draxin polypeptide.

6. A composition comprising a plurality of nanoparticles, wherein each nanoparticle comprises at least two polypeptides selected from the group consisting of a IL-4 polypeptide, a IL-10 polypeptide, a TGFß polypeptide, a IGF1 polypeptide, a draxin polypeptide, a NPTX2 polypeptide, a PTN polypeptide, a TGFB3 polypeptide, a PRXL2A polypeptide, a MMP13 polypeptide, a MMP9 polypeptide, and a ADAM22 polypeptide.

7. The composition of claim 6, wherein each nanoparticle comprises said IL-10 polypeptide, said TGFß polypeptide, said IGF1 polypeptide, and said draxin polypeptide.

8. The composition of claim 6, wherein each nanoparticle comprises said IL-4 polypeptide, said IL-10 polypeptide, said TGFß polypeptide, said IGF1 polypeptide, and said draxin polypeptide.

9. A method for treating a mammal having a stroke, wherein said method comprises administering the composition of claim 1.

10. The method of claim 9, wherein said administering is performed while said mammal is experiencing said stroke.

11. A method for treating a mammal having had a stroke, wherein said method comprises administering the composition of claim 1.

12. The method of claim 11, wherein said administering is performed within from about 1 minute to about 72 hours of the mammal experiencing the stroke.

13. The method of claim 9, wherein said mammal is a human.

14. The method of claim 9, wherein said stroke is selected from the group consisting of an ischemic stroke, a hemorrhagic stroke, and a ministroke.

15. The method of claim 9, wherein said method is effective to reduce or eliminate a symptom of stroke in said mammal.

16. The method of claim 15, wherein said symptom is selected from the group consisting of slurring words, confusion, paralysis of the face, arm, or leg, numbness of the face, arm, or leg, weakness of the face, arm, or leg, problems seeing, headache, stumbling, losing balance when walking, dizziness, and loss of coordination.

17. The method of claim 9, wherein said method is effective to improve sensorimotor function in said mammal.

18. The method of claim 9, wherein said method is effective to improve cognitive function in said mammal.

19. The method of claim 9, wherein said method is effective to improve psychiatric function in said mammal.
